(19) United States
(12) Patent Application Publication
(10) Pub. No.: US 2013/0296672 A1
(43) Pub. Date: Nov. 7, 2013

(54) NONINVASIVE PHYSIOLOGICAL SENSOR COVER

(71) Applicant: MASIMO CORPORATION, Irvine, CA (US)

(72) Inventors: Charles D. O'Neil, Mission Viejo, CA (US); John Schmidt, Huntington Beach, CA (US); Nicholas Evan Barker, Laguna Beach, CA (US)

(73) Assignee: Masimo Corporation, Irvine, CA (US)

(21) Appl. No.: 13/875,219

(22) Filed: May 1, 2013

Related U.S. Application Data
(60) Provisional application No. 61/641,611, filed on May 2, 2012.

(51) Int. Cl. A61B 5/1455; A61B 5/0205

(52) U.S. Cl. CPC A61B 5/14552; A61B 5/0205
USPC 600/324; 600/323

(57) ABSTRACT

A sensor cover according to embodiments of the disclosure is capable of being used with a noninvasive physiological sensor, such as a pulse oximetry sensor. Certain embodiments of the sensor cover reduce or eliminate false readings from the sensor when the sensor is not in use, for example, by blocking a light detecting component of a pulse oximeter sensor when the pulse oximeter sensor is active but not in use. In certain embodiment, the sensor cover has a pattern that allows it to be more easily seen on a surface such as a floor. Further, embodiments of the sensor cover prevent contamination of the sensor. Additionally, embodiments of the sensor cover can prevent damage to the sensor.

### CROSS-REFERENCE TO RELATED APPLICATIONS

This application claims the benefit under 35 U.S.C. §119(e) as a nonprovisional of U.S. Provisional Application No. 61/641,611, filed May 2, 2012, titled NON-INVASIVE PHYSIOLOGICAL SENSOR COVER, the entirety of which is incorporated herein by reference.

### FIELD OF THE DISCLOSURE

The present disclosure relates to a sensor for measuring oxygen content in the blood, and, in particular, relates to an apparatus and method for preventing sensor activity when the sensor is not in use.

### BACKGROUND OF THE DISCLOSURE

Noninvasive physiological sensors are applied to the body for monitoring or making measurements indicative of a patient's health. One application for a noninvasive physiological sensor is pulse oximetry, which provides a noninvasive procedure for measuring the oxygen status of circulating blood. Oximetry has gained rapid acceptance in a wide variety of medical applications, including surgical wards, intensive care and neonatal units, general wards, and home care and physical training. A pulse oximetry system generally includes a patient monitor, a communications medium such as a cable, and a physiological sensor having light emitters and a detector, such as one or more LEDs and a photodetector. The sensor is attached to a tissue site, such as a finger, toe, ear lobe, nose, hand, foot, or other site having pulsatile blood flow which can be penetrated by light from the emitters. The detector is responsive to the emitted light after attenuation by pulsatile blood flowing in the tissue site. The detector outputs a detector signal to the monitor over the communication medium, which processes the signal to provide a numerical readout of physiological parameters such as oxygen saturation (SpO2) and pulse rate.

High fidelity pulse oximeters capable of reading through motion induced noise are disclosed in U.S. Pat. Nos. 6,770,028, 6,658,276, 6,157,850, 6,002,952 5,769,785, and 5,758,644, which are assigned to Masimo Corporation (“Masimo”) and are incorporated by reference herein. Advanced physiological monitoring systems may incorporate pulse oximetry in addition to advanced features for the calculation and display of other blood parameters, such as carboxyhemoglobin (HbCO), methemoglobin (HbMet) and total hemoglobin (Hbt), total Hematocrit (Hct), oxygen concentrations and glucose concentrations, as a few examples. Advanced physiological monitors and corresponding multiple wavelength optical sensors capable of measuring parameters in addition to SpO₂, such as HbCO, HbMet and Hbt are described in at least U.S. patent application Ser. No. 11/367,013, filed Mar. 1, 2006, titled Multiple Wavelength Sensor Emitters and U.S. patent application Ser. No. 11/366,208, filed Mar. 1, 2006, titled Noninvasive Multi-Parameter Patient Monitor, assigned to Masimo Laboratories, Inc. and incorporated by reference herein. Further, noninvasive blood parameter monitors and optical sensors including Rainbow™ adhesive and reusable sensors and RAD57™ and Radical-7™ monitors capable of measuring SpO₂, pulse rate, perfusion index (PI), signal quality (SiQ), pulse variability index (PVI), HbCO and HbMet, among other parameters, are also commercially available from Masimo.

### SUMMARY OF THE DISCLOSURE

Optical sensors are widely used across clinical settings, such as operating rooms, emergency rooms, post anesthesia care units, critical care units, outpatient surgery and physiological labs, to name a few. In some situations, such as in operating rooms, emergency rooms or critical care units, sensors can be kept attached to monitors to reduce the setup time needed to begin monitoring a patient. While attached, the sensor can generate false readings by detecting ambient light even though the sensor is not in use. The sensor can also cause the monitor to emit alarms or otherwise make noise due to false readings, which can be distracting to medical personnel.

As such, a method and apparatus for preventing false readings are desirable. A sensor cover, according to embodiments of the disclosure, prevents or reduces false readings until the sensor is in use. In certain embodiment, the sensor cover has a pattern that can be easily seen on a surface such as a floor of a clinical setting.

Further, in certain embodiments, a sensor cover decreases the likelihood of contamination by keeping covered portions of the sensor clean. Sensors in hospitals and other clinical environments are subject to exposure to infectious agents, dust, or other foreign matter from depositing on sensor components. The sensor cover can reduce or prevent exposure to these contaminants. In some embodiments, the sensor cover can prevent damage to the sensor. For example, the sensor covers can protect the sensor components during shipment or prior to use.

### BRIEF DESCRIPTION OF THE DRAWINGS

FIG. 1 illustrates a physiological measurement system according to an embodiment of the disclosure;

FIG. 2 illustrates an embodiment of a perspective view of an optical sensor of FIG. 1;

FIG. 3A illustrates an embodiment of a sensor cover over an optical sensor;

FIG. 3B illustrates a top-down view of the embodiment of the sensor cover of FIG. 3A;

FIG. 4A illustrates another embodiment of a sensor cover over an optical sensor;

FIG. 4B illustrates a top-down view of the embodiment of the sensor cover of FIG. 4A; and

FIG. 5 illustrates an embodiment of a sensor cover partially removed from an optical sensor.

### DETAILED DESCRIPTION

A sensor cover according to embodiments of the disclosure is capable of being used with a noninvasive physiological sensor. Certain embodiments of the sensor cover reduce or eliminate false readings from the sensor when the sensor is not in use. Some embodiments of the sensor cover can have patterns that aid seeing the sensor cover on a floor of a clinical settings, such as operating rooms, emergency rooms, post anesthesia care units, critical care units, outpatient surgery and physiological labs, to name a few. Further, embodiments of the sensor cover prevent contamination of the sensor. Additionally, embodiments of the sensor cover can prevent damage to the sensor.

The tissue site of the illustrated embodiments is a finger and the following description therefore refers specifically to the tissue site as a finger for the purposes of clarity. This is not intended to be limiting and, as described herein, the sensor cover of certain embodiments can be used with sensors attachable to other types of tissue sites, such as a toe, ear lobe, nose, hand, foot, forehead, or the like.

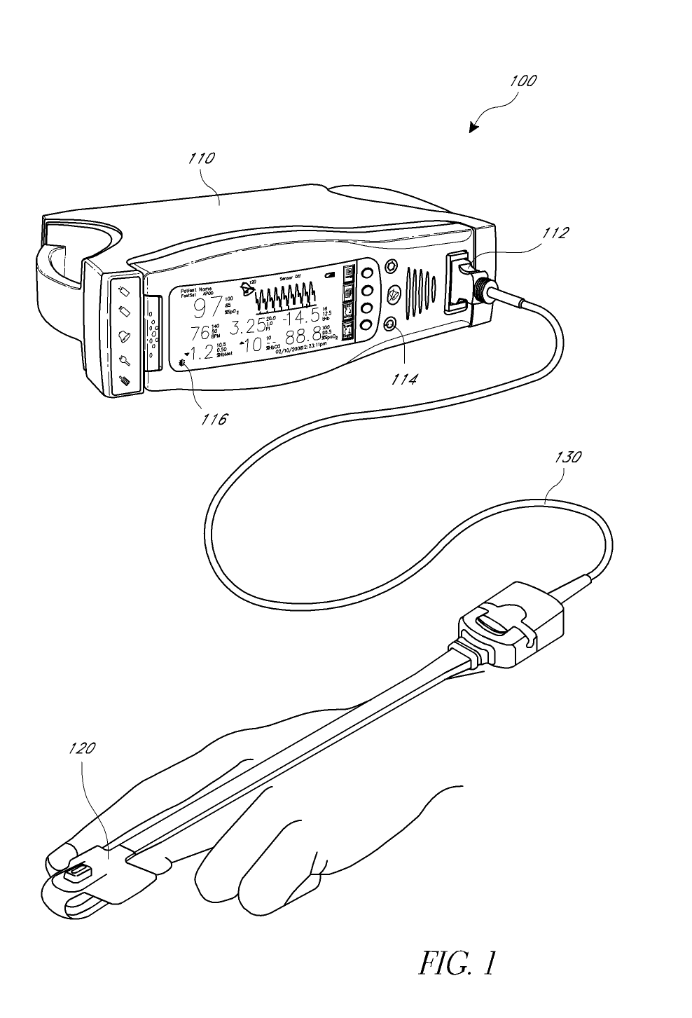

FIG. 1 illustrates an embodiment of an optical sensor attached to a physiological measurement system 100 having a monitor 110 and an optical sensor 120. The optical sensor 120 comprises one or more light emitters and a detector. The optical sensor 120 is configured to plug into a monitor sensor port 112 via a patient cable 130. Monitor keys 114 provide control over operating modes and alarms, to name a few. A display 116 provides readouts of measured parameters, such as oxygen saturation, pulse rate, HbCO, HbMet, and Hbt, to name a few. Other blood parameters that can be measured to provide important clinical information are fractional oxygen saturation, bilrubin, and blood glucose, to name a few.

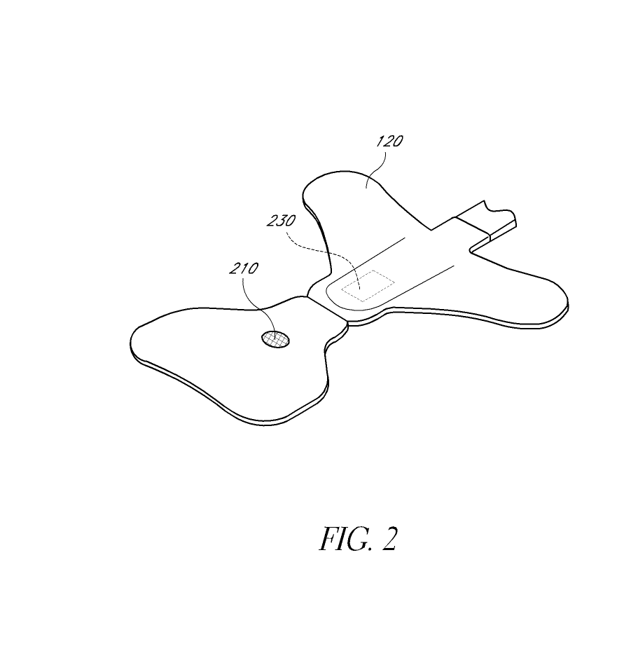

FIG. 2 illustrates an embodiment of a side view of an optical sensor 120 not attached to a finger or another tissue site. The optical sensor 120 comprises one or more light emitters 230 and a detector 210.

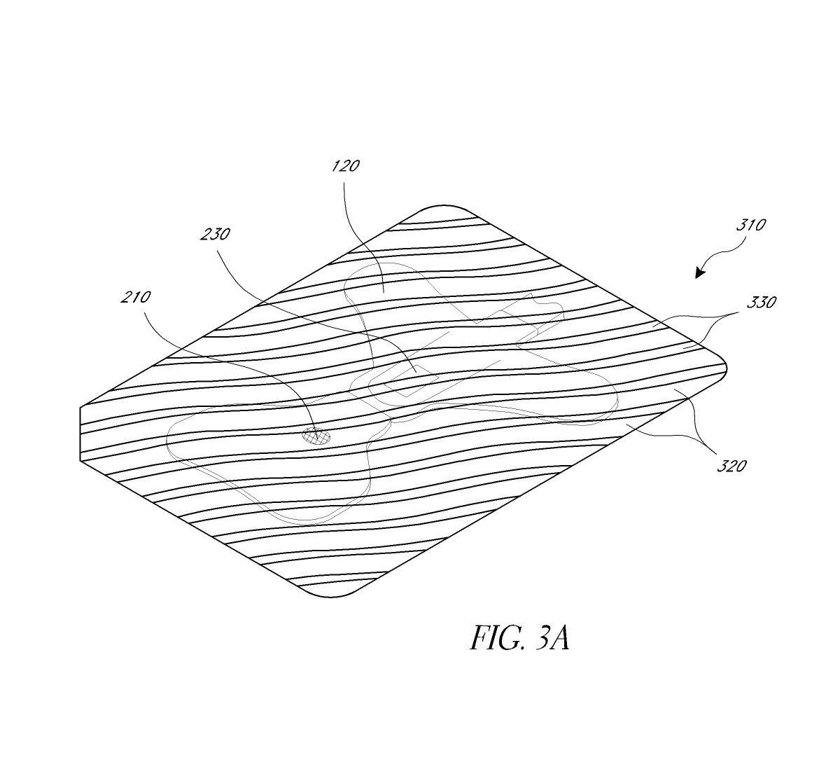

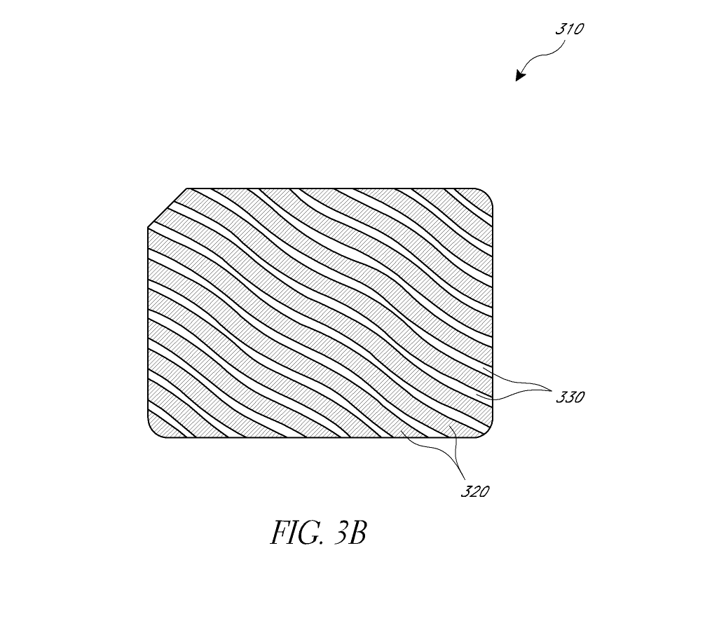

In the illustrated embodiment of FIG. 3A, a sensor cover 310 covers the optical sensor 120. The sensor cover 310 of FIG. 3A illustrates dark portions 320 and lighter portions 330 being the same opaqueness and/or color. The same opaqueness and/or color is for illustration purposes to show the overlay of the sensor cover 310 over the optical sensor 120. The contrast and/or pattern formed by the dark portions 320 and the lighter portions 330 is illustrated in FIG. 3B and discussed herein. The cover can be made from a clear, semi-opaque, and/or opaque material, such as, for example, plastic, polyester, polypropylene, rubber, vinyl, cling vinyl, and/or the like. In the illustrated embodiment, the sensor cover 310 obstructs the detector 210 and prevents the detector 210 from detecting light, thereby reducing or eliminating false readings by covering the detector 210 with a dark portion 320 as described herein.

The optical sensor 120 can sometimes be left attached to a monitor 110 to facilitate quick monitoring of a patient, even when not currently in use. The sensor cover 310 can prevent or reduce false readings caused by the emitters 230 or the ambient light, even if the sensor is active, by preventing the detector 210 from receiving light. In certain embodiment, the sensor cover 310 can be placed over the optical sensor 120 such that the dark portions 320 are over the emitters 230, preventing the emitters 230 from emitting light receivable by the detector 210.

FIG. 3B is a top-down view of the sensor cover 310 embodiment of FIG. 3A. While the overall shape of the sensor cover 310 is illustrated in FIG. 3B as substantially rectangular, the sensor cover 310 can be square, trapezoidal, triangular, round, oval, and/or the like. As illustrated in FIG. 3B, the sensor cover 310 has dark portions 320 and lighter portions 330. The dark portions 320 can cover the detector 210 and prevent the detector 210 from detecting light. The dark portions 320 can cover the emitters 230 and prevent the emitters 230 from emitting light. In an embodiment, the dark portion 320 can block all wavelengths of light used by a particular sensor. The dark portions 320 can be opaque. In some embodiments, the dark portion 320 can block different ranges of wavelengths depending on the type of sensor the cover is used for. The dark portion 320 can be semi-opaque. The lighter portions 330 can be semi-opaque, but more transparent than the opaque and/or semi-opaque dark portions 320. In some embodiments, the lighter portions 330 are clear.

In certain embodiments, the dark portions 320 and lighter portions 330 form a pattern. The pattern can be striped. The striped pattern can be formed as straight stripes. The stripes can be evenly spaced apart and/or irregularly spaced apart. The darker stripes formed by the darker portions 320 can be smaller, same, and/or larger in size in comparison to the lighter stripes formed by the lighter portions 330. The striped pattern can be wave-like, wavy, sinuous, etc. The striped pattern can be wave-like, but irregular. The striped pattern can have step-like transitions. The step-like transitions can be regular and/or irregular. Some embodiments of the sensor cover 310 may have a striped pattern that is a combination of straight, wavy, irregular wavy, regular step-like, and/or irregular step-like transitions. In some embodiments, the dark portions 320 and the lighter portions 330 can form a pattern comprising shapes such as circles, triangles, squares, polygons, and/or the like. Either the dark portions 320 or the lighter portions 330 can form the shapes on the sensor cover 310. The shapes can be the same and/or different size. The shapes can be placed in a regular and/or irregular pattern on the sensor cover 310. Some embodiments of the sensor cover 310 may have a combination of the striped patterns and the shape patterns described herein. In certain embodiments, the pattern on the sensor cover 310 is designed such that when the sensor cover 320 is placed on the optical sensor 120, the dark portions 310 cover at least one of either the detector 210 or the emitters 230. The design of the pattern is spaced such that the dark portions 310 cover at least one of either the detector 210 or the emitters 230 no matter the orientation or placement of the sensor cover 310 on the optical sensor 120 as long as the optical sensor 120 is fully covered by the sensor cover 310.

In some embodiments, the dark portions 320 can be printed onto the sensor cover 310 that has an initial configuration of having the lighter portions 330. In some embodiments, the dark portions 320 can be dyed into the sensor cover 310 that has an initial configuration of having the lighter portions 330. Some embodiments are coextruded and/or laminated to form the sensor cover 310 with a pattern of dark portions 320 and lighter portions 330. Some embodiments can be manufactured using 3D printing or additive manufacturing to create a sensor cover 310 with a pattern of dark portions 320 and lighter portions 330. In certain embodiments, the sensor cover 310 can also be fabricated using any suitable or known process or processes, including injection molding, compression molding, and/or thermoforming techniques to form a pattern of dark portions 320 and lighter portions 330.

The contrast in opaqueness between the dark portions 320 and the lighter portions 330 and/or the patterns described herein can be such that when the sensor cover 310 is placed against a surface either with or removed from the optical sensor 120, the sensor cover 310 creates a contrast with the surface that is easily seen by a user, such as a medical personnel or a patient. The surface can be a floor of a clinical settings, such as operating rooms, emergency rooms, post anesthesia care units, critical care units, outpatient surgery and physiological labs, to name a few. The surface can also be, for example, a countertop, or tabletop. Part of the surface may be seen through either the darker portions 320 and/or the lighter portions 330. Less of the surface can be seen through the darker portions 320 than the lighter portions 330, creating a contrast and/or a unique pattern when placed against the surface. The contrast and/or unique pattern can attract a user's attention. Attracting a user's attention can help prevent the user from slipping or falling and/or damaging the optical sensor 120 by stepping on the sensor cover 310 and/or optical sensor 120 when the sensor cover 310 is located on, for example, the floor of a hospital room or other clinical environments. If the surface is a countertop or tabletop in, for example, an ambulance vehicle, a user aware of the sensor cover 310 can avoid placing equipment, such as the physiological measurement monitor 110, on the sensor cover 310 and/or optical sensor 120 to help prevent damage to the optical sensor 120 and/or to help better secure the monitor 110 to the surface when the ambulance vehicle brakes, corners, and/or accelerates.

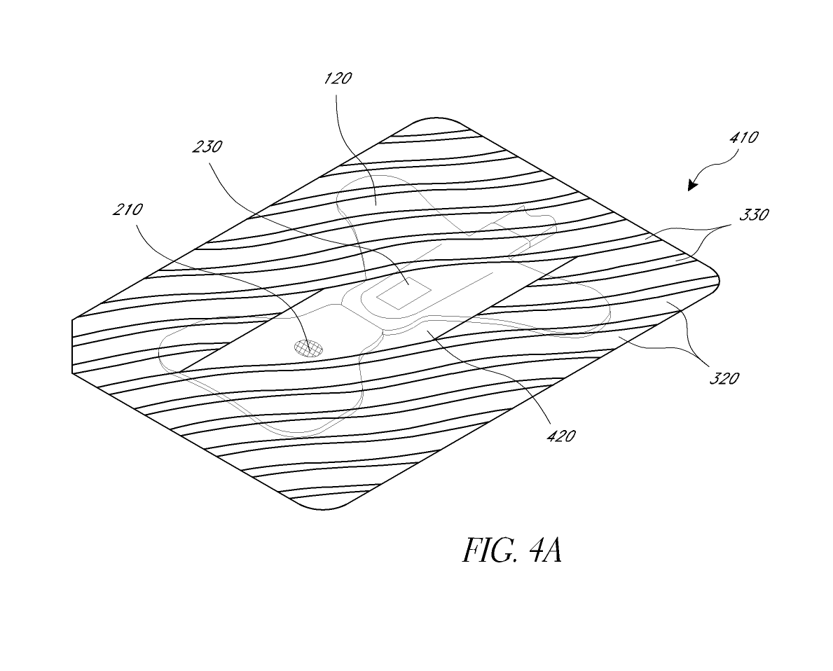

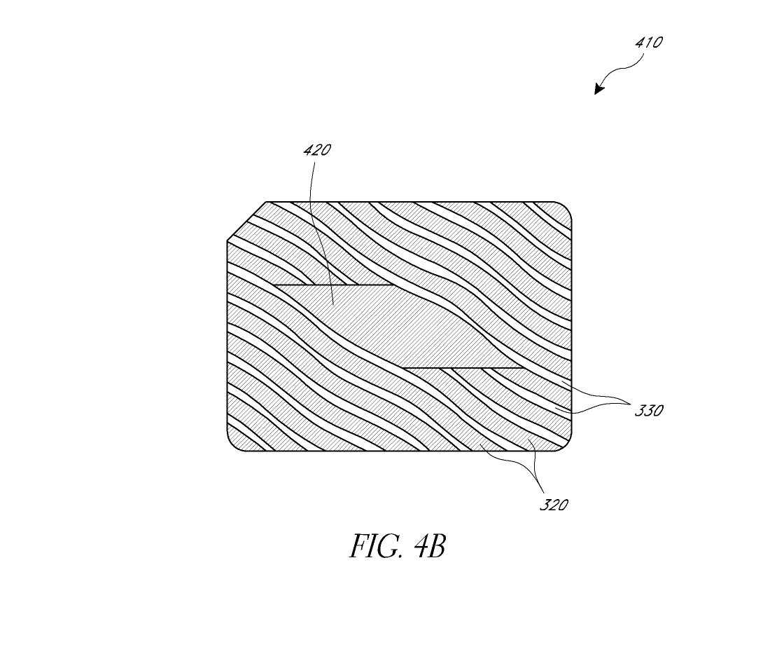

In the illustrated embodiment of FIGS. 4A and 4B, a sensor cover 410 covers the optical sensor 120. The sensor cover 410 of FIG. 4A illustrates dark portion 420, dark portions 320, and lighter portions 330 being the same opaqueness and/or color. The same opaqueness and/or color is for illustration purposes to show the overlay of the sensor cover 410 over the optical sensor 120. The contrast and/or pattern formed by the dark portions 320 and 420 and the lighter portions 330 is illustrated in FIG. 4B and discussed herein. The sensor cover 410 can be made of the same materials, manufactured using the same methods, and/or patterned the same way as described for the sensor cover 310 embodiment of FIGS. 3A and 3B. The illustrated embodiment of FIG. 4A has a dark portion 420 covering both the detector 210 and the emitters 230. The dark portion 420 can be any shape or a combination of shapes such as a circle, triangle, square, polygon, and/or the like. The dark portion 420 can extend from side to side of the sensor cover 410 along the sensor cover's 410 width and/or length. The dark portion 420 can extend from corner to corner of the sensor cover 410. The dark portion 420 can be part of or integrated as part of the pattern formed, as described herein, by the dark portions 320 and lighter portions 330. The dark portion 420 can be the same as and/or different opaqueness than the dark portions 320. The sensor cover 410 can be shipped from the manufacturer with the dark portion 420 covering both the detector 210 and the emitters 230. Upon removal of the sensor cover 410 and reapplication of the sensor cover 410, the pattern formed by the dark portions 320 and 420 and the lighter portions 330 is spaced such that the dark portions 320 and 420 cover at least one of either the detector 210 or the emitters 230 no matter the orientation or placement of the sensor cover 410 on the optical sensor 120 as long as the optical sensor 120 is fully covered by the sensor cover 410.

In some embodiments, the sensor cover 310, 410 is removed before placement at a tissue or measurement site. For example, once a patient arrives, medical personnel can remove the sensor cover 310, 410 and attach the now fully operational sensor 120 to the patient. The sensor cover 310, 410 can be removed by peeling it off from the sensor 120.

In the illustrated embodiments of FIGS. 3A-B and 4A-B, the sensor cover 310, 410 placed over the optical sensor 120 can decrease the likelihood of contamination by keeping the optical sensor 120 covered until application to a measurement site. Sensors in hospitals and other clinical environments are subject to exposure to infectious agents, dust, or other foreign matter from depositing on the emitters or detector. The sensor cover 310, 410 can reduce or prevent exposure to these contaminants. In certain embodiments, the sensor cover 310, 410 can prevent damage to the sensor 120. For example, the sensor cover 310, 410 can protect the detector 210 and the emitters 230 during shipment or prior to use. The detector 210 and the emitters 230 are protected by the avoidance of direct contact with foreign matter until the sensor cover 310, 410 is removed.

As will be appreciated by skilled artisans from the disclosure provided herein, various attachment mechanisms can be used. For example, the sensor cover can be attached with an adhesive. In certain embodiments, a restorable adhesive can be used to facilitate reattachments of the sensor cover. The restorable adhesive layer can be rejuvenated by application of alcohol to the adhesive. The cover can then be reattached to the sensor. This can be useful where the sensor is moved to a new location or tissue site because the cover can prevent the sensor from taking false readings while the sensor is moved. In some embodiments, no adhesive is used on the sensor cover to leave no residue. In some embodiments, the sensor cover can be made from static cling vinyl, plastic film, or other “clingy” material with no adhesive used. In some embodiments, the sensor cover can be attached through static electricity allowing the cover to cling to the sensor without any adhesive and/or allowing the sensor cover to be reapplied. In other configurations, the sensor cover can be attached with Velcro, fasteners, tabs, clips, slots, or the like.

In certain embodiments, the sensor covers are reusable. For example, the sensor cover can be reused if the sensor is temporarily removed for repositioning or for cleaning. The sensor cover can also be replaced on the sensor when the sensor is no longer in use. In some embodiments, the sensor covers are disposable and are disposed of once removed from the sensor.

Although disclosed with reference to the sensor of FIG. 1, an artisan will recognize from the disclosure herein a wide variety of oximeter sensors, optical sensors, noninvasive sensors, medical sensors, or the like that may benefit from the sensor cover disclosed herein. In various embodiments, the sensor can be adapted to receive a tissue site other than a finger such as a, toe, ear lobe, nose, hand, foot, neck, or other site having pulsatile blood flow which can be penetrated by light from the emitter. In addition, the sensor cover can be used with a portable monitor and associated sensor components in certain embodiments. Such monitors, including the sensor components, can be integrated into a hand-held device such as a PDA and typically do not include cables or separate monitors. Portable monitors are often used by first responders in emergency situations, in part because of their portability and ease of use. As such, sensor covers which can protect the sensor components according to embodiments herein can be of particular benefit when used with spot-check monitors.

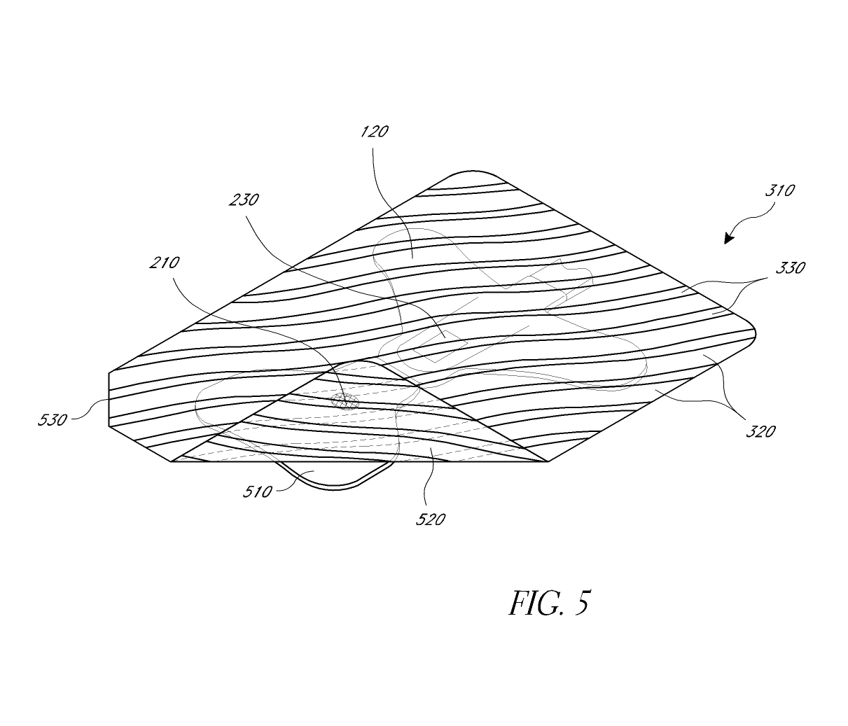

FIG. 5 illustrates a sensor cover 310 placed over the optical sensor 120 to cover the detector 210 and emitters 230. In an embodiment, the optical sensor 120 has an adhesive layer on a cover side 510 to attach to the sensor cover 310 and/or measurement site. The sensor cover 310 can have a smooth surface on the sensor side 520 to facilitate the removal the optical sensor 120 from the sensor cover 310. The smooth surface can be made from the same material as the sensor cover 310. In some embodiments, the smooth surface can be an additional layer. The additional layer material can be any suitable material that does not cling to the adhesive layer on the cover side 510, such as silicone, rubber, polyethylene, etc. The sensor cover 310 can have a cut-off corner 530 to identify the sensor side 520 so that the smooth surface is facing the optical sensor 120 during application. The sensor cover 310 can be peeled off to reveal the cover side 510 with the adhesive layer and to uncover the sensor components, such as the detector 210 and the emitters 230.

In some embodiments, the sensor side 520 of the sensor cover 310 can include an adhesive layer over the portion of the cover designed to attach to the optical sensor 120 at cover side 510 while the remainder of the sensor cover 310 can be adhesive free. Thus, the sensor cover 310 does not catch on other objects and cause the sensor cover 310 to be prematurely removed. The sensor cover 310 can be removed by peeling the optical sensor 120 off the sensor cover 310. The sensor cover 310 can have a cut-off corner 530 to identify the sensor side 520 so that the adhesive layer is facing the optical sensor 120 during application.

Although the above embodiments have been described with respect to an opaque material intended to optically insulate the optical sensor, as will be appreciated by skilled artisans from the disclosure provided herein, sensor covers made of different insulative materials can be used as appropriate for different types of sensors. For example, sonically insulative materials, such as foam, rubber, cotton, and/or other sound deadening materials can be used to cover sensors that employ sound, such as a bioacoustic or ultrasound sensor. In some embodiments, electrically insulative materials, such as rubber, polyethylene, silicone, and/or other insulators can be used to cover sensors that employ electrical signals, such as bioimpedance sensors. In some embodiments, mechanically insulative materials, such as hard plastic, metal, rubber, silicone, and/or other rigid or dampening materials can be used to cover mechanical sensors to prevent sensor actuation. In some embodiments, chemically insulative material, such as plastic, metal, polyethylene or the like can be used to cover chemical sensors and prevent their exposure to the environment.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements, and/or states are in any way required for one or more embodiments or that one or more embodiments necessarily include logic for deciding, with or without author input or prompting, whether these features, elements, and/or states are included or are to be performed in any particular embodiment.

Various sensor covers have been disclosed in detail in connection with various embodiments. These embodiments are disclosed by way of examples only and are not to limit the scope of the claims that follow. One of ordinary skill in the art will appreciate the many variations, modifications, and combinations. For example, in various embodiments, adhesive, snap-fit, friction-fit, clips, tabs, and other attachment mechanisms can be employed. In addition, in various embodiments the sensor covers are used with a sensor that can measure any type of physiological parameter. In various embodiments, the sensor covers can be for any type of medical device or sensor. In various embodiments, adhesive can be placed on both sides of the sensor cover to aid in the reattachment of sensors where the sensor adhesive has grown weak. In various embodiments, sensors covers can be made in whole or in part of materials such as foam, polyester, polypropylene, rubber, vinyl, cling vinyl, urethane rubber plastic, or other plastic materials, cloth, metal, combinations of the same or the like.

What is claimed is:

1. A sensor covering system for a noninvasive physiological sensor, the sensor covering system comprising:
a noninvasive physiological sensor comprising sensing components including a light-emitting component and a light-detecting component; and
a sensor cover configured to cover the noninvasive physiological sensor in a first configuration;
the sensor cover comprising a pattern with a dark portion preventing, in the first configuration, measurement of one or more physiological parameters by the noninvasive physiological sensor while the noninvasive physiological sensor is active; and
wherein the removal of the sensor cover in a second configuration allows the noninvasive physiological sensor to actively measure the one or more physiological parameters in the second configuration.

2. The noninvasive physiological sensor of claim 1, further comprising an adhesive layer on a cover side the noninvasive physiological sensor.

3. The sensor cover of claim 1, further comprising a sensor side having a smooth surface configured to allow removal of the noninvasive physiological sensor.

4. The sensor cover of claim 1, further comprising an identification feature to identify sides of the sensor cover.

5. The sensor cover of claim 4, wherein the sensor cover is substantially rectangular, and wherein the identification feature comprises a cut-off corner where one of the corners of the rectangular sensor cover is removed.

6. The sensor covering system of claim 1, wherein the dark portion covers at least one of the sensing of components in the first configuration.

7. The sensor covering system of claim 1, wherein the dark portion is semi-opaque.

8. The sensor covering system of claim 1, wherein the dark portion is opaque.

9. The sensor covering system of claim 1, wherein the pattern of the sensor cover further comprises a lighter portion.

10. The sensor covering system of claim 9, wherein the lighter portion is semi-opaque.

11. The sensor covering system of claim 9, wherein the lighter portion is clear.

12. The sensor covering system of claim 9, wherein the dark portion and the lighter portion substantially form a striped arrangement.

13. The sensor covering system of claim 12, wherein the striped arrangement substantially forms a wave pattern.

14. The sensor covering system of claim 1, wherein the pattern of the sensor cover is configured to be in contrast to a surface of a room containing the sensor cover when the sensor cover is placed on the surface.

15. A method of preventing a noninvasive physiological sensor having a detector for measuring a physiological variable, the method comprising:
providing a sensor cover with a pattern, the pattern configured to block light; and
attaching the sensor cover to the noninvasive physiological sensor, the pattern covering the detector to prevent the detector from receiving light, wherein blocking the light prevents the detector from taking a measurement, and the sensor cover removable from the detector.

16. The method of claim 15, wherein the light is ambient light from a surrounding area.

17. The method of claim 15, wherein the noninvasive physiological sensor is a pulse oximeter sensor.

18. The method of claim 15 further comprising:
activating a light source of the noninvasive physiological sensor to emit light from one or more emitters of the sensor; and
blocking the light from the one or more emitters from being received by the detector with the sensor cover.

19. The method of claim 18 further comprising:
removing the sensor cover from the noninvasive physiological sensor; and
attaching the noninvasive physiological sensor to a patient.

20. The method of claim 19 further comprising:
removing the noninvasive physiological sensor from the patient; and
reattaching the sensor cover to the noninvasive physiological sensor to cover either the detector or the one or more emitters.

21. A method of preventing a noninvasive physiological sensor having one or more emitters for emitting light and a detector for measuring a physiological variable, the method comprising:
providing a sensor cover with a pattern, the pattern configured to block light; and
attaching the sensor cover to the noninvasive physiological sensor, the pattern covering at least one of the one or more emitters to prevent the emitter from emitting light or the detector to prevent the detector from receiving light, wherein blocking the light prevents the detector from taking a measurement, and the sensor cover removable from the detector.

22. The method of claim 21, wherein the light to the detector includes light from the one or more emitters and ambient light from a surrounding area.

23. The method of claim 21, wherein the noninvasive physiological sensor is a pulse oximeter sensor.

24. The method of claim 21 further comprising:
removing the sensor cover from the noninvasive physiological sensor; and
attaching the noninvasive physiological sensor to a patient.

25. The method of claim 24 further comprising:
removing the noninvasive physiological sensor from the patient; and
reattaching the sensor cover to the noninvasive physiological sensor to cover at least one of the one or more emitters or the detector.

26. The method of claim 21, wherein the pattern covers the one or more emitters to prevent the emitter from emitting light.

27. The method of claim 21, wherein the pattern covers the detector to prevent the detector from receiving light.
